(19) United States
(12) Patent Application Publication
(10) Pub. No.: US 2017/0058308 A1
(43) Pub. Date: Mar. 2, 2017

(54) INTEGRATED CONTINUOUS BIOMANUFACTURING PROCESS

(71) Applicant: Novo Nordisk A/S, Bagsvaerd (DK)

(72) Inventors: Mats Aakesson, Hoellviken (SE); Martin Heitmann, Copenhagen (DK); Peter Tiainen, Limhamn (SE)

(21) Appl. No.: 15/306,938

(22) PCT Filed: Apr. 30, 2015
(86) PCT No.: PCT/EP2015/059594

§ 371 Date: Oct. 26, 2016

(30) Foreign Application Priority Data
May 2, 2014 (EP) 14166896.2

(51) Int. Cl. C12P 21/00; C07K 1/16; C07K 14/755; B01D 15/36; C07K 16/00; C07K 14/62; B01D 15/38; C12M 1/00; C12N 9/64

(52) U.S. Cl. CPC C12P 21/00; C12M 29/10; C12M 27/18; C12M 47/12; C12M 47/10; C07K 1/16; C07K 14/755; C12N 9/6437; C07K 16/00; C07K 14/62; C12Y 304/21021; B01D 15/3804; B01D 15/363

(57) ABSTRACT

The invention is a process and apparatus thereto for the end-to-end and continuous production of a purified protein, the process comprising the use of an integrated apparatus comprising a first and second processing unit having continuous and matched outflow and inflow, thereby providing for a means of integration of a protein cultivation system and chromatographic systems.

### TECHNICAL FIELD

The invention relates to an integrated process and apparatus thereto for the continuous production of a purified protein.

### BACKGROUND

Large-scale, economic purification of proteins is increasingly an important problem for the biotechnology and pharmaceutical industry. Typically, proteins are produced by cell culture, using either mammalian, yeast or bacterial cell lines engineered to produce the protein of interest by insertion of a recombinant plasmid containing the gene for that protein. Since the cell lines used are living organisms, they must be fed with a complex growth medium, containing sugars, amino acids, and growth factors. Separation of the desired protein from the mixture of compounds fed to the cells and from the by-products of the cells themselves to a purity sufficient for use as a human therapeutic poses a formidable challenge. The biopharmaceutical industry is constantly searching for cost-effective, flexible manufacturing strategies that can live up to strict product quality requirements. In this context, automated continuous manufacturing processes have the potential to provide attractive bioprocessing solutions by offering high volumetric productivity and steady-state operation with consistent product quality. A strategy integrating upstream perfusion cultivation and downstream continuous capture has previously been reported (Warikoo et al., Integrated continuous production of recombinant therapeutic proteins, Biotechnol. Bioeng. 109: 3018-3029 (2012))

Process intensification through conversion from batch to continuous manufacturing has long been applied in several industries and there is now a clear interest awakening in the bioprocessing industry. Key drivers are steady-state operation, short residence and processing times, streamlined process flows and high volumetric productivity. Reduction of equipment size and removal of intermediate hold steps allow for minimized facilities which yields significantly reduced capital costs.

Within biotech, continuous processing also has the potential to provide advantages wrt to protein quality. Reduced processing time and elimination of intermediate hold steps decreases the target protein's exposure to enzymatic, chemical and physical degradation/modification and thereby increases protein quality.

When connecting two independent process units operating continuously, there is a challenge of matching their flows, i.e., the outflow of the upstream unit should match that of the downstream unit (e.g. to avoid process failure due to overflow/overpressure or gas bubbles).

A common solution is to introduce an intermediate surge vessel acting as a buffer between the two units. For instance Warikoo et al. (Biotechnol. Bioeng. 2012; 109:3018-3029) describes perfusion cultivation integrated with a continuous capture chromatography unit by means of an intermediate surge bag. WO 2006/039588 describes examples where surge vessels are used to integrate continuous clarification with either ultrafiltration or semi-continuous rapid chromatography (Vogel et al., Biotechnol. Bioeng. 2012; 109: 3049-3058).

U.S. Pat. No. 4,630,639 discloses two systems connected by a passage, which in turn has a constant flow control valve and a constant pressure control valve, wherein the first system can be a hydraulic source and the second system can be a cylinder.

WO 2011/037522 relates to a separation system comprising two separation units wherein the separation units are connected in series outlet to inlet to form a line of separation units using; and sensing and adjustment means, provided in-line between each separation unit, for continuously monitoring and adjusting at least one environmental property parameter of fluid flowing from one separation unit to a subsequent separation unit in the line of separation units.

The present investigation allows for an integrated system without the use of a surge vessel since such devices are problematic for proteins with stability issues. The processing time of a target molecule from its expression to purification or isolation is reduced by the present method.

### SUMMARY

The present invention is directed, in one aspect, to a continuous processing concept realising a combined up- and downstream end-to-end continuous bioprocessing platform.

When connecting two independent process units in a continuous process, there is a challenge of matching their flows, i.e., the outflow of the preceding unit should match that of the following unit. By the present invention, an apparatus and method has been developed that avoids the conventional step of introducing an intermediate storage/surge vessel which increases the processing time and increases the protein's exposure to enzymatic, chemical and physical degradation/modification. It is an object of the present invention to provide an improved automated separation system which is capable of further reducing development time, processing time and cost of goods and which does not require intermediate holding tanks.

A first aspect of the invention is directed to an apparatus comprising
a. at least two independent processing units, a first processing unit and a second processing unit, each processing unit comprising a fluid inlet, a fluid outlet, and a fluid delivery device, where the first and second processing units are connected in series by at least one fluid connection with fluid flowing from outlet of one processing unit to inlet of a subsequent processing unit, and
b. at least one means for introducing additional liquid to the fluid connection
c. at least one means for removing excess liquid from the fluid connection

A further aspect of the invention is directed to an apparatus, comprising
a. at least two independent processing units, a first and a second processing unit, each processing unit comprising a fluid inlet, a fluid outlet, and a fluid delivery device, where the first and the second processing units are connected in series by at least one fluid connection with fluid flowing from outlet of one processing unit to inlet of a subsequent processing unit;
b. at least one fluid inlet in the fluid connection between two processing units
c. at least one fluid outlet in the fluid connection between two processing units.

A related aspect of the invention is directed to a method of recombinant protein production from a cell line comprising the steps of

i. cultivating a cell line in a cultivation unit where an outflow of culture supernatant is provided by a pump connecting the cultivation unit to a (automated) purification unit using an apparatus of the invention;

ii. performing a protein purification on said (automated) purification unit with an feed inflow provided by a pump.

The invention is furthermore directed to a method for separating a target protein or proteins from a heterogeneous fluid mixture comprising
i. a first processing step comprising producing a fluid mixture containing the protein of interest
ii. transferring by means of a unidirectional flow the fluid mixture from an outlet of a first processing step to an inlet of a second processing step (without use of intermediate holding vessel)
iii. matching the flow rate of liquid delivered from first processing step to that of flow rate of liquid taken up in second processing step by removing surplus liquid or adding compatible liquid
iv. a second processing step comprising producing a further purified fluid mixture containing the protein of interest.

Another related aspect of the invention is directed to a method for separating a protein of interest from a heterogeneous fluid mixture comprising
i. producing, by a first processing step, a fluid mixture containing the protein of interest
ii. transferring the fluid mixture from an outlet of a first processing step to an inlet of a second processing step comprising matching the flow rate of liquid delivered from first processing step to that of flow rate of liquid taken up in a second processing step by removing surplus liquid or adding compatible liquid
iii. producing, by said second processing step, a further purified fluid mixture containing the protein of interest.

Notably, the transferring and matching is step ii) is without use of intermediate holding vessel.

The invention is furthermore directed to a process for the continuous or semi-continuous production of a purified protein, the process comprising the use of a an integrated apparatus comprising
a. a cell culture bioreactor with cell separation unit having continuous outflow
b. a means for at least partially purifying a protein with a continuous inflow, such as a means for performing chromatography or a means for filtration,
c. a device with two three-way connectors and two check valves for matching the outflow of the bioreactor and the inflow of the means for at least partially purifying a protein.

### BRIEF DESCRIPTION OF DRAWINGS

FIG. 1 depicts an embodiment of the invention comprising first processing unit, (a) System 1 pump, a second processing unit, (b) System 2 pump, check valves (c) and (d), and a flow (e) of e.g. buffer solution and (f) surplus flow to e.g. collection tank.

FIG. 2 depicts a further configuration of an embodiment of the invention comprising a first processing unit, (a) System 1 pump, a second processing unit, (b) System 2 pump, check valves (c) and (d), and a flow (e) of e.g. buffer solution and (f) surplus flow to e.g. collection tank.

FIG. 3 depicts a still further configuration of an embodiment of the invention comprising a first processing unit, (a) System 1 pump, a second processing unit, (b) System 2 pump, check valves (c) and (d), and a flow (e) of e.g. buffer solution and (f) surplus flow to e.g. collection tank.

FIG. 4 depicts a configuration of an embodiment of the invention comprising a first processing unit, (a) System 1 pump, a second processing unit, (b) System 2 pump, check valves (c) and (d), additional flow from System 1 (e) and (f) surplus flow to e.g. collection tank.

FIG. 5 depicts a setup for an embodiment of the invention comprising a protein perfusion production with integrated continuous purification, (a) a fresh medium supply, (b) a feed pump (peristaltic pump—controlled by level sensor), (c) a cell culture bioreactor, (d) an ATF cell retention device, (e) a bleed pump (peristaltic pump—controlled by biomass/capacitance signal), (f) a cell discard vessel, (g) a 0.22 μm absolute filter, (h) a harvest pump (peristaltic pump—set to defined perfusion rate), (i) a check valve 1, (j) an ÄKTA sample application pump, (k) a parallel capture columns, (l) a check valve 2, (m) a vessel, (n) buffer flasks, (o) an ÄKTA gradient pump, (p) a mixer, (q) a waste vessel, (r) a column 3 and 5, (s) a column 2, 4 and 6, (t) a UV and conductivity detectors and (u) a final purified protein pool.

FIG. 6 depicts the FVIII concentration measured by RP-HPLC in final gel filtration pools in Example 1.

FIG. 7 depicts a SDS-PAGE of final gel filtration pools in Example 1.

FIG. 8 depicts an example of UV chromatogram from final gel filtration step in Example 2.

FIG. 9 depicts the period of integrated operation in Example 2. Left panel: Viable cell density (VCD). Right panel: Estimates (based on gel filtration chromatograms) of purified dimer and dimer fraction in corresponding sub-batches.

FIG. 10 depicts the period of integrated operation in Example 2. Left panel: Estimates (based on gel filtration chromatograms) of purified dimer and dimer fraction in corresponding sub-batches. Right panel: Selected gel filtration chromatograms, before, during and after change in cultivation conditions.

FIG. 11 depicts, in the integrated continuous capture of FVII variant, the capture yields for period of integrated operation in Example 3.

FIG. 12 depicts, in the integrated continuous capture of FVII variant, the viable cell density (VCD), viability, and titer for ATF perfusion cultivation in Example 3. The grey area indicates the period of integrated continuous cultivation and capture.

FIG. 13 depicts the weight increase in collection tank during continuous operation in Example 4 with inflow of the ÄKTA purification system reduced to 75% of initial flow rate.

FIG. 14 depicts SE-HPLC chromatograms for yield evaluation of sub-batches in Example 4. Shown are sub-batches run at 100% (A), 75% (B) and 125% (C) of initial ÄKTA purification system in-flow rate.

FIG. 15 depicts product quality evaluation by SDS-PAGE of monoclonal antibody sub-batches run in Example 4. Represented sub-batches were run at 100% (A), 75% (B) and 125% (C) of initial ÄKTA purification system in-flow rate.

FIG. 16 depicts the setup for an up-stream and down-stream integrated insulin precursor production. Legend (a) Yeast growth medium supply, (b) Feed pump, (c) Bioreactor, (d) peristaltic pump, (e) Collection vessel, (f) Double-headed peristaltic pump, (g) Waste bottle, (h) Cross-flow filtration vessel, (i) Cross-flow filtration 0.2 μm filter, (j) Cross-flow filtration pump (TMP controlled), (k) Dilution buffer, (l) Buffer flask with check valve 1, (m) Vessel with check valve 2, (n) Piston pump with in-line dilution, (o) Dilution buffer, (p) Buffers [e.g. 100 mM Tris buffer pH 8 (elution buffer)], (q) Gradient piston pumps, (r) Mixer, (s) Parallel capture columns, (t) Waste flask, (u) UV and conductivity detectors and (v) final purified protein pool

FIG. 17 depicts the SDS-PAGE of recovered insulin precursor. Lane A. comprises of insulin precursor and lane B. of ALP digested insulin precursor. The marker is SeeBlue Plus2 and it is non-reduced conditions.

FIG. 18 depicts a cross-flow filtration device coupled to an ÄKTA having (1.) a buffer flask with a check valve only allowing flow out from that flask and (2.) a surplus collection tank with a check valve only allowing flow into that tank in-between them. Both flasks were put on balances. The ÄKTA flow was set initially to 11 ml/min (100%).

### DESCRIPTION

Continuous process designs offer several advantages including streamlined process flows and high volumetric productivities. This enables a reduction of equipment size and removal of intermediate holding steps which in turn allow for compact facilities and reduced capital costs. For bioprocessing, a reduced overall processing time and elimination of intermediate storage also decreases the product's exposure to enzymatic, chemical and physical modifications. This makes continuous processing particularly attractive for the production of fragile proteins.

As part of the present invention, we have developed an integrated continuous framework for end-to-end production of complex fragile proteins based on perfusion cultivation and automated multi-step purification. Upstream, the integrated system may consist of a stirred tank bioreactor with an ATF cell retention system. Automatic feedback control of viable biomass using an on-line capacitance probe ensures robust long-term operation at steady-state and hence a constant and consistent product stream for downstream processing. The clarified harvest may directly enter a filtration or chromatography system, such any conventional chromatography system, for example but without limitation an ÄKTA chromatography system, employed as a continuous purification unit. Two alternating capture columns may precede a multi-step purification train with full flexibility and control of individual columns.

The integrated set-up provides a compact automated bench-top factory converting cell culture media to purified protein in an efficient manner without intermediate storage. It reduces the lead time from start of expression to purified protein compared to traditional batch-wise processing. Furthermore, the integrated approach also provides continuous monitoring of the process allowing for “just-enough” production and better use of resources.

The target molecule or molecules refers to any molecule, substance or compound or mixtures thereof that shall be isolated, separated or purified from one or more impurities in a sample. The target molecule or molecules is/are typically a protein, a nucleic acid sequence or nucleotide, such as a DNA or RNA sequence. Proteins, such as those part of a sample from the first processing unit which have been subject to expression typically to purification in a second processing unit. In a preferred embodiment, the target molecule is a protein or a mixture of two or more proteins. In a very preferred embodiment, the target molecule is an antibody or a coagulation factor, such as Factor V, VII, VIII, IX, X, and XIII, or conjugates and variants thereof. The target molecule may also be selected from a coagulation factor and fusion or conjugate of a coagulation factor with a protein such as an antibody or fragment thereof, with a albumin binder such as a fatty acid chain, or with a protein such as albumin. The target protein may alternatively be selected from insulin, variants thereof, and enzymes used in the processing of insulin, such is trypsin or a lysyl specific protease, such as Achromobacter lyticus protease.

The term “antibody” refers to a protein which has the ability to specifically bind to an antigen. Typically, antibodies are having a basic four-polypeptide chain structure consisting of two heavy and two light chains, said chains being stabilized, for example, by interchain disulfide bonds. Antibodies may be monoclonal or polyclonal and may exist in monomeric or polymeric form, for example, IgM antibodies which exist in pentameric form and/or IgA antibodies which exist in monomeric, dimeric or multimeric form. Antibodies may also include multispecific antibodies (e.g., bispecific antibodies), and antibody fragments so long as they retain, or are modified to comprise, a ligand-specific binding domain. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. When produced recombinantly, fragments may be expressed alone or as part of a larger protein called a fusion protein. Exemplary fragments include Fab, Fab′, F(ab′)2, Fc and/or Fv fragments. Exemplary fusion proteins include Fc fusion proteins. According to the present invention, fusion proteins are also encompassed by the term “antibody”.

As discussed above, in some embodiments, an antibody is an Fc region containing protein, e.g., an immunoglobulin. In some embodiments, an Fc region containing protein is a recombinant protein which includes the Fc region of an immunoglobulin fused to another polypeptide or a fragment thereof. Exemplary polypeptides include, e.g., renin; a growth hormone, including human growth hormone and bovine growth hormone; growth hormone releasing factor; parathyroid hormone; thyroid stimulating hormone; lipoproteins; a-1-antitrypsin; insulin, insulin a-chain; insulin [beta]-chain; proinsulin; and enzymes used in the processing of insulin, such is trypsin or a lysyl specific protease, such as Achromobacter lyticus proteasefollicle stimulating hormone; calcitonin; luteinizing hormone; glucagon; clotting factors such as Factor V, Factor VII, Factor VIII, Factor IX, Factor X, Factor XIII, Tissue Factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumour necrosis factor-a and -beta; enkephalinase; RANTES (regulated on activation normally T-cell expressed and secreted); human macrophage inflammatory protein (MIP-1-a); a serum albumin such as human serum albumin; Muellerian-inhibiting substance; relaxin a-chain; relaxin [beta]-chain; prorelaxin; mouse gonadotropin-associated peptide; a microbial protein, such as [beta]-lactamase; DNase; IgE; a cytotoxic T-lymphocyte associated antigen (CTLA) (e.g., CTLA-4); inhibin; activin; vascular endothelial growth factor (VEGF); receptors for hormones or growth factors; Protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-[beta].; platelet-derived growth factor (PDGF); fibroblast growth factor (FGF); epidermal growth factor (EGF); transforming growth factor (TGF) such as TGF-alpha and TGF-[beta], TGF-2, TGF-3, TGF-4, or TGF-[beta][delta]; insulin-like growth factor-I and -II (IGF-I and IGF-II); des(I-3)-IGF-I (brain IGF-I), insulin-like growth factor binding proteins (IGFBPs); CD proteins such as CD3, CD4, CD8, CD 19 CD20, CD34, and CD40; erythropoietin; osteoinductive factors; immunotoxins; a bone morphogenetic protein (BMP); an interferon such as interferon-[alpha], -[beta], and -[gamma]; colony stimulating factors (CSFs), e.g., M-CSF, GM-CSF, and G-CSF; interleukins (ILs), e.g., IL-I to IL-IO; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CDI Ia, CDI Ib, CDI Ic, CD 18, an ICAM, VLA-4 and VCAM; a tumour associated antigen such as HER2, HER3 or HER4 receptor; and fragments and/or variants of any of the above-listed polypeptides. In addition, an antibody according to the present invention is any protein or polypeptide, fragment or variant thereof that binds specifically to any of the above-listed polypeptides.

In a suitable embodiment, the first processing unit is a cultivation unit with a continuous out-flow, such as a perfusion cultivation unit. A cultivation unit is any system for cultivation of suspension or adherent cells such as a perfusion culture system, and typically comprising cells, and a cell media formulation. The terms cell cultivation, cell perfusion or cell perfusion cultivation are intended to mean any system where a host cell or organism produces a target protein, such as a system with harvested cell culture fluid comprising the target protein. Embodiments of the first processing unit include cell perfusion systems such as bioreactors and fermenters equipped with a cell retention system. An example of such a cell retention system is the ATF™ system based upon the technology of Alternating Tangential Flow, created by the action of a diaphragm moving upwards and then downwards within a pump head, connected to a filter housing and attached to a standard bioreactor. The first processing unit, such as a cultivation unit, provides a sample for processing by the second processing unit.

The term “sample” refers to any composition or mixture that contains a target molecule. As discussed, the samples may be derived from biological or other sources from the first processing unit. Biological sources include eukaryotic and prokaryotic sources, such as plant and animal cells, tissues and organs. The sample may also include diluents, buffers, detergents, and contaminating species, debris and the like that are found mixed with the target molecule.

Typically, one or both of the processing units involve purifying the target molecule.

The terms “purifying,” “separating,” or “isolating,” as used interchangeably herein, refer to increasing the degree of purity of a target molecule from a composition or sample comprising the target molecule and one or more impurities. Typically, the degree of purity of the target molecule is increased by removing (completely or partially) at least one impurity from the composition.

The term “chromatography” refers to any kind of technique which separates an analyte of interest (e.g. a target molecule) from other molecules present in a mixture. Usually, the target molecule is separated from other molecules as a result of differences in rates at which the individual molecules of the mixture migrate through a stationary medium under the influence of a moving phase, or in bind and elute processes. The term “matrix” or “chromatography matrix” are used interchangeably herein and refer to any kind of sorbent, resin or solid phase which in a separation process separates a target molecule (e.g., an Fc region containing protein such as an immunoglobulin) from other molecules present in a mixture. Non-limiting examples include particulate, monolithic or fibrous resins as well as membranes that can be put in columns or cartridges. Examples of materials for forming the matrix include polysaccharides (such as agarose and cellulose); and other mechanically stable matrices such as silica (e.g. controlled pore glass), poly(styrenedivinyl)benzene, polyacrylamide, ceramic particles and derivatives of any of the above. Examples for typical matrix types suitable for the method of the present invention are cation exchange resins, affinity resins, anion exchange resins or mixed mode resins. A “ligand” is a functional group that is attached to the chromatography matrix and that determines the binding properties of the matrix. Examples of “ligands” include, but are not limited to, ion exchange groups, hydrophobic interaction groups, hydrophilic interaction groups, thiophilic interactions groups, metal affinity groups, affinity groups, bioaffinity groups, and mixed mode groups (combinations of the aforementioned). Some preferred ligands that can be used herein include, but are not limited to, strong cation exchange groups, such as sulphopropyl, sulfonic acid; strong anion exchange groups, such as trimethylammonium chloride; weak cation exchange groups, such as carboxylic acid; weak anion exchange groups, such as N5N diethylamino or DEAE; hydrophobic interaction groups, such as phenyl, butyl, propyl, hexyl; and affinity groups, such as Protein A, Protein G, and Protein L.

The term “affinity chromatography” refers to a protein separation technique in which a target protein (e.g., an Fc region containing protein of interest or antibody) is specifically bound to a ligand which is specific for the target protein. Such a ligand is generally referred to as a biospecific ligand. In some embodiments, the biospecific ligand (e.g., Protein A or a functional variant thereof) is covalently attached to a chromatography matrix material and is accessible to the target protein in solution as the solution contacts the chromatography matrix. The target protein generally retains its specific binding affinity for the biospecific ligand during the chromatographic steps, while other solutes and/or proteins in the mixture do not bind appreciably or specifically to the ligand. Binding of the target protein to the immobilized ligand allows contaminating proteins or protein impurities to be passed through the chromatography matrix while the target protein remains specifically bound to the immobilized ligand on the solid phase material. The specifically bound target protein is then removed in active form from the immobilized ligand under suitable conditions (e.g., low pH, high pH, high salt, competing ligand etc.), and passed through the chromatographic column with the elution buffer, free of the contaminating proteins or protein impurities that were earlier allowed to pass through the column. Any component can be used as a ligand for purifying its respective specific binding protein, e.g. antibody. However, in various methods according to the present invention, Protein A is used as a ligand for an Fc region containing target protein. The conditions for elution from the biospecific ligand (e.g., Protein A) of the target protein (e.g., an Fc region containing protein) can be readily determined by one of ordinary skill in the art. In some embodiments, Protein G or Protein L or a functional variant thereof may be used as a biospecific ligand. In some embodiments, a biospecific ligand such as Protein A is used at a pH range of 5-9 for binding to an Fc region containing protein, washing or re-equilibrating the biospecific ligand/target protein conjugate, followed by elution with a buffer having pH about or below 4 which contains at least one salt.

As stated, one aspect of the invention is directed to an apparatus comprising
a. at least two independent processing units, a first processing unit and a second processing unit, each processing unit comprising a fluid inlet, a fluid outlet, and a fluid delivery device, where the first and second processing units are connected in series by at least one fluid connection with fluid flowing from outlet of one processing unit to inlet of a subsequent processing unit;
b. at least one means for introducing additional liquid to the fluid connection; and
c. at least one means for removing excess liquid from the fluid connection.

Alternatively defined, the apparatus typically comprises
a. at least two independent processing units, a first processing unit and a second processing unit, each processing unit comprising a fluid inlet, a fluid outlet, and a fluid delivery device, where the first and second processing units are connected in series by at least one fluid connection with fluid flowing from outlet of one processing unit to inlet of a subsequent processing unit;
b. at least one fluid inlet in the fluid connection between two processing units, providing a means for introducing additional liquid to the fluid connection; and
c. at least one fluid outlet in the fluid connection between two processing units, providing a means for removing excess liquid from the fluid connection

The flow direction of the inlet is suitably restricted, by means of unidirectional flow control, to restrict the liquid to flow unidirectionally towards the fluid connection between two processing units. The inlet of said fluid connection preferably has a flow direction restricted by a means of unidirectional flow control to restrict the liquid to flow unidirectionally towards the fluid connection between processing units.

Alternatively or in combination, the flow direction of the outlet may be restricted by means of unidirectional flow control to restrict liquid to flow unidirectionally from the fluid connection between processing units. Preferably, the outlet said fluid connection has a flow direction restricted by a means of unidirectional flow control to restrict liquid to flow unidirectionally from the fluid connection between processing units. The means of restricting the unidirectional flow of the inlet or the outlet is typically a check valve.

In various embodiments, at least one of the fluid delivery devices of the apparatus comprises a pump. Both or one of the fluid delivery devices may comprise a pump.

The apparatuses of the invention are typically intended for use for recombinant protein production from a cell line. Accordingly, a further aspect of the invention is directed to a method of recombinant protein production from a cell line comprising the steps of

i. cultivating a cell line in a cultivation unit where an outflow of culture supernatant is provided by a pump connecting the cultivation unit to a purification unit using any apparatus defined by the invention; and

ii. performing a protein purification on said purification unit with an feed inflow provided by a pump. The purification unit is typically an automated purification unit known to the practitioner.

Without limitation, the cell line may be from a prokaryotic or eukaryotic cell but is typically a mammalian cell line. The cell line may selected from the group consisting of be yeast, a bacterial cell line and a eukaryotic cell line. Typical bacterial cell line may be selected from Escherichia coli, B. subtilis, Corynebacterium, and Pseudomonas fluorescens. The eukaryotic cells lines may be further selected from S. cerevisiae and also those of the Bacillus gender, Pichia pastoris, and Filamentous fungi, such as Aspergillus, Trichoderma, Myceliophthora thermophila. The cell line may furthermore be from Baculovirus-infected cells, non-lytic insect cell expression insect cells, or mammalian cells (HeLa, HEK 293). The cell line may be from plant systems, such as tobacco but also tomato, lettuce, carrot plants and transplastomic plants, such as those comprising chloroplast expression vectors. The cell line may be from mammalian systems including bovine (such as Bos primigenius), mouse (such as Mus musculus), Chinese Hamster Ovary, Baby Hamster Kidney and Human Embryonic Kidney cells.

Without limitation, the target protein may be a polypeptide included glycopeptides. Interesting embodiments are wherein the protein is selected from the group consisting an antibody, a coagulation factor, such as Factor V, VII, VIII, IX, X, and XIII, or variants thereof, a soluble receptor, a growth hormone, and insulin or variants thereof, particularly wherein the protein is selected from the group consisting of a coagulation factor and an antibody. The protein purification is typically performed by means of filtration or chromatography.

An aspect of the invention is directed to an apparatus for separating a molecule of interest from a heterogeneous fluid mixture comprising
a. at least two independent processing units, a first and a second processing unit, each processing unit comprising a fluid inlet, a fluid outlet, and a fluid delivery device, where the first and the second processing units are connected in series by at least one fluid connection with fluid flowing from outlet of one processing unit to inlet of a subsequent processing unit;
b. at least one fluid inlet in the fluid connection between two processing units.
c. at least one fluid outlet in the fluid connection between two processing units.

The first processing unit may be typically selected from the group consisting of a bioreactor, a fermentation unit, a tube reactor, an ultrafiltration unit, a homogenizer, a centrifuge unit, and a chromatography unit, each of which with a continuous or semi-continuous outflow, preferably a continuous outflow. The second processing unit is typically selected from the group consisting of an ultrafiltration unit, a homogenizer, a centrifuge unit, and a chromatography unit, each of which with a continuous or semi-continuous outflow, preferably a continuous inflow.

In a combination of suitable embodiments, the first processing unit is a cultivation unit with a continuous out-flow, such as a perfusion cultivation unit, and the second process unit is a chromatography system with a continuous in-flow.

In a further combination of suitable embodiments, the first processing unit is an ultrafiltration unit with a continuous out-flow and the second process unit is a chromatography system with a continuous in-flow.

In a combination of suitable embodiments wherein the first processing unit is a chromatography system with a continuous or semi-continuous out-flow, such as an Äkta chromatography system and the second process unit is an ultrafiltration unit with a continuous in-flow.

In a further combination of suitable embodiments, the first processing unit is a simulated moving bed chromatography system with a continuous out-flow, and the second process unit is a chromatography system with a continuous in-flow, such as an Äkta chromatography system.

In another combination of suitable embodiments, the first processing unit is a chromatography system with a continuous or semi-continuous out-flow, such as an Äkta chromatography system, and the second process unit is a chromatography system with a continuous in-flow, such as an Äkta chromatography system.

In yet a further combination of suitable embodiments, the first processing unit is a tube reactor with a continuous or semi-continuous out-flow and the second process unit is a chromatography system with a continuous in-flow, such as an Äkta chromatography system.

In yet a further combination of suitable embodiments, the first processing unit is a homogenizer with a continuous out-flow and the second process unit is a continuous centrifuge with a continuous in-flow.

In yet a further combination of suitable embodiments, the first processing unit is a continuous fermentation with a continuous out-flow and the second process unit is a continuous centrifuge with a continuous in-flow.

In another combination of suitable embodiments, the first processing unit is a continuous centrifuge with a continuous out-flow and the second process unit is a chromatography system with a continuous in-flow, such as an Äkta chromatography system.

In each of the embodiments, semi-continuous outlet flows may be matched with continuous in-let flows. For instance, the outlet flow from a first processing unit, such as homogeniser may be in pulses into a continuous second processing unit such as a centrifuge. The invention provides that the reduced flow between pulses is provided for, or “the blanks are filled in” and flows matched.

A further aspect of the invention is directed to a method for purifying a liquid containing at least one target molecule, comprising the use of an apparatus as defined herein.

An interesting aspect of the invention is directed a method for separating a target protein or proteins from a heterogeneous fluid mixture comprising
i. a first processing step comprising producing a fluid mixture containing the protein of interest
ii. transferring by means of a unidirectional flow a fluid mixture from an outlet of a first processing step to an inlet of a second processing step (without use of intermediate holding vessel)
iii. matching the flow rate of liquid delivered from first processing step to that of flow rate of liquid taken up in second processing step by removing surplus liquid or adding compatible liquid
iv. a second processing step comprising producing a further purified fluid mixture containing the protein of interest.

In some embodiments of the invention, it may be preferred that the fluid mixture produced by the first processing step is partially purified, such as by filtration, before transferring the fluid mixture to the inlet of the second processing step.

In an exemplary embodiment of the invention, the first processing step is a continuous cultivation process producing clarified cell culture harvest fluid containing the protein of interest; and the second processing step is a continuous chromatography process producing partially purified fluid containing the target protein or proteins.

The invention is further directed to a process for the continuous or semi-continuous production of a purified protein, the process comprising the use of a an integrated apparatus comprising
a. a cell culture bioreactor with cell separation unit having continuous outflow
b. a means for at least partially purifying a protein with a continuous inflow, such as a means for performing chromatography or a means for filtration,
c. a device with two three was connectors and two check valves for matching the outflow of the bioreactor and the inflow of the means for at least partially purifying a protein.

The upstream part of the integrated system, the first processing unit, may comprise a bioreactor operating in perfusion mode, using an ATF cell retention system. Feedback control of viable cell concentration using an on-line capacitance probe may be applied to ensure robust long-term operation at steady state. Downstream, an ÄKTA follows with an in-line dilution option of the application leading to two alternating chromatography capture columns before a flexible and well-controlled multi-step purification system. The option of having in-line dilution of the stream from the bioreactor (i.e. pH adjustment, addition of salts etc.) widens the choice of capture method. Down-stream process monitoring is accomplished by having detectors in the flow path between column valves. Moreover, buffer exchange columns are used to adjust parameters like conductivity and/or pH between individual column steps allowing for automation of purification processes with various column combinations. All together this makes the end-to-end setup very flexible.

The embodiment wherein the first processing step is perfusion cultivation and the second processing step comprises continuous multi-step purification enables flexible and highly productive manufacturing units. Furthermore, removal of hold steps or surge vessels minimises the risk for unwanted protein degradation which makes it ideally suited for production of complex unstable proteins. Consequently, the end-to-end continuous manufacturing strategy will be exemplified with a complex recombinant protein expressed in CHO cells using a chemically defined medium.

In one embodiment of the invention, the present invention combines a multi-step purification with full control between columns and the feasibility of continuous operation a chromatography system, such as on ÄKTA chromatography system. By combining these two aspects a basic chromatography system, such as the ÄKTA system, can be transformed into a continuous purification unit. The presented setup is based on an off-the-shelf solution, without customised parts like personalised software strategies, thereby making automated and continuous chromatography widely available.

When connecting two independent process units in a continuous process, there is a challenge of matching their flows, i.e., the outflow of the preceding unit should match that of the following unit. In the prior art, this has been solved by introducing an intermediate storage/surge vessel. This introduces an unnecessary storage which increases the processing time and increases the protein's exposure to enzymatic, chemical and physical degradation/modification.

When connecting a semi-continuous process to a continuous process, there is also a challenge of matching flows, with prior art solutions with storage/surge vessels. The surge vessels again introduce unnecessary storage which increases the total process time and reduces protein stability.

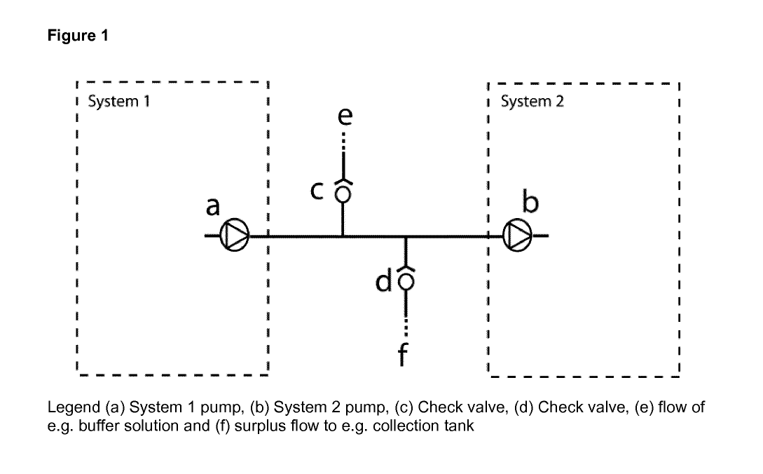

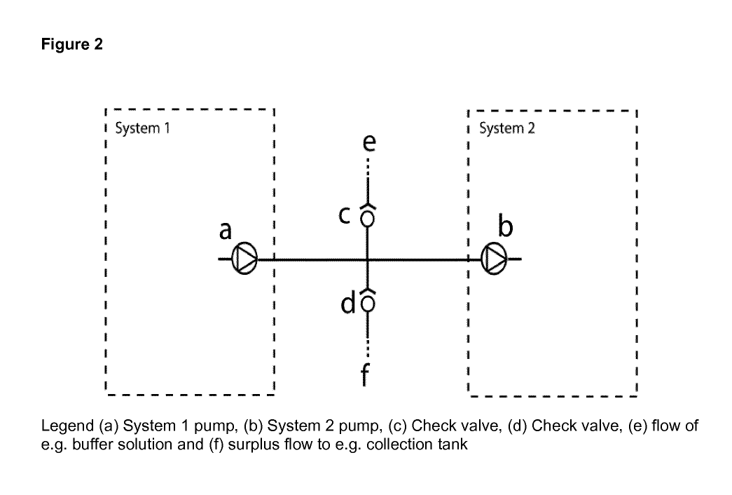

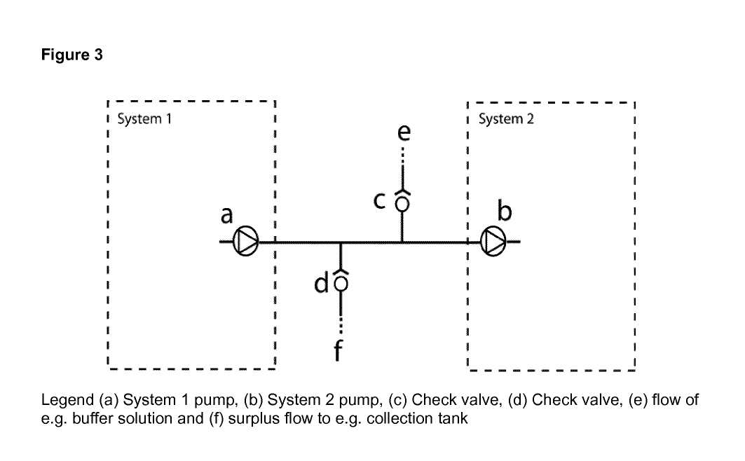

According to a typical embodiment of the invention, a direct connection between two continuous processing units, or one semi-continuous unit connected with a continuous unit, without intermediate surge/storage vessel employing an arrangement with two three-way connectors and two check valves (see FIGS. 1-3 for different configurations). If the flow delivered from the first processing unit is lower than that required by second processing unit, the difference flow (e) will be delivered via the check valve (c) connected to e.g. a supply of a compatible buffer solution. If the flow delivered from the first processing unit is higher than that taken up by the second processing unit, the surplus flow (f) will go through the check valve (d) to e.g. a collection tank.

If the flow from the first processing unit is semi-continuous, delivered in batches, the liquid needed to sustain a flow during the idle periods of the first processing unit, where the flow from the first processing unit is zero, will be delivered via the check valve (c) connected to e.g. a supply of a compatible buffer solution.

In this way, there is no additional storage and processing time introduced in the process.

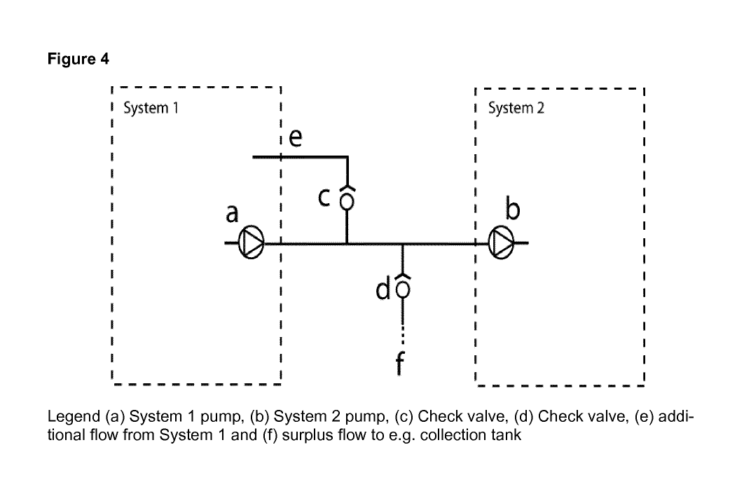

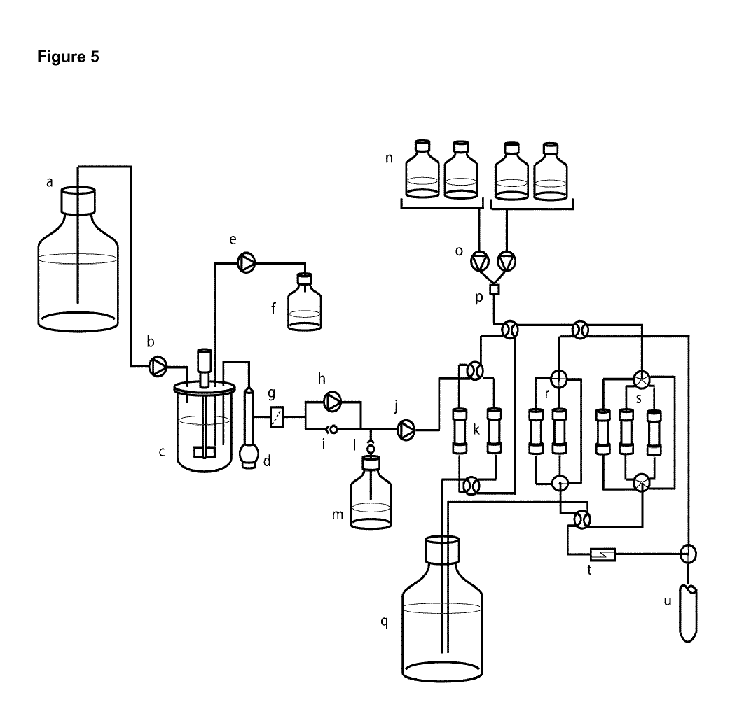

Variants of the generic configurations depicted in FIGS. 1-3 may include configurations where the difference and/or the surplus flows are coming from the first processing unit and/or the second processing unit. For instance, see FIG. 4 where the difference flow (e) is coming from System 1. One particular implementation of this can be seen in FIG. 5 where an ATF perfusion cultivation system is connected to an ÄKTA purification system. If the flow delivered by the ATF harvest pump (h) is lower than the flow taken by the ÄKTA pump (j), the difference flow will be taken via the check valve (i). If the flow delivered from the ATF pump is higher than that taken up by the ÄKTA pump (j), the surplus flow will go through the check valve (l) to e.g. a collection vessel (m).

As discussed, the method of the invention involves combining into an apparatus at least a first and a second processing units apparatuses where processing units are connected in series by at least one fluid connection with fluid flowing from outlet of one processing unit to inlet of a subsequent processing unit. Individually, each processing unit is an independent processing unit.

The first processing unit is typically selected from the group consisting of a bioreactor, a fermentation unit, a tube reactor, an ultrafiltration unit, a homogenizer, a centrifuge unit, and a chromatography unit, each of which with a continuous or semi-continuous outflow, preferably a continuous outflow.

The second processing unit is typically selected from the group consisting of an ultrafiltration unit, a homogenizer, a centrifuge unit, and a chromatography unit, each of which with a continuous or semi-continuous outflow, preferably a continuous inflow.

In one embodiment, the first processing unit is a cultivation unit with a continuous out-flow, such as a perfusion cultivation unit, and the second process unit is a chromatography system with a continuous in-flow, such as an Äkta chromatography system. This embodiment is illustrated, in a non-limiting manner, in FIG. 5.

In another suitable embodiment, the first processing unit is an ultrafiltration unit with a continuous out-flow and the second process unit is a chromatography system with a continuous in-flow, such as an Äkta chromatography system.

In another suitable embodiment, the first processing unit is a chromatography system with a continuous or semi-continuous out-flow, such as an Äkta chromatography system and the second process unit is an ultrafiltration unit with a continuous in-flow.

In another suitable embodiment, the first processing unit is a simulated moving bed chromatography system with a continuous out-flow, and the second process unit is a chromatography system with a continuous in-flow, such as an Äkta chromatography system.

In another suitable embodiment, the first processing unit is a chromatography system with a continuous or semi-continuous out-flow, such as an Äkta chromatography system, and the second process unit is a chromatography system with a continuous in-flow, such as an Äkta chromatography system.

In another suitable embodiment, the first processing unit is a tube reactor with a continuous or semi-continuous out-flow and the second process unit is a chromatography system with a continuous in-flow, such as an Äkta chromatography system.

In another suitable embodiment, the first processing unit is a homogenizer with a continuous out-flow and the second process unit is a continuous centrifuge with a continuous in-flow.

In another suitable embodiment, the first processing unit is a continuous fermentation with a continuous out-flow and the second process unit is a continuous centrifuge with a continuous in-flow.

In another suitable embodiment, the first processing unit is a continuous centrifuge with a continuous out-flow and the second process unit is a chromatography system with a continuous in-flow, such as an Äkta chromatography system.

The invention may be described as generally directed to the coupling of two continuous process steps into an integrated process for the growth/synthesis/preparation of a product (unstable or inconvenient to store temporarily) by means of a device which performs the desired function (or in the absence of intermediate storage device).

Aspects of the invention may be related to coupling into an integrated system, a cell culture bioreactor with ATF module to a set of columns for purification of the protein, both being continuous in flow processes, as well as coupling two continuous in flow process steps without limitation on the type of continuous in flow process methods (cultivation, filtration, chromatography, homogenization, centrifugation).

More specifically, the device may be directed to a device for use in the preparation and purification of a protein (without limitation on the type of protein).

Unstable proteins, such as FVIII, are specifically enabled but the device provides at least some of its advantages in the preparation and purification of all proteins, including coagulation factors, insulin, GLP derivatives, GHs, receptors, antibodies/FAbs, etc.

The following embodiments are preferred modes of performing the invention:
1. An apparatus comprising
   a. at least two independent processing units, a first processing unit and a second processing unit, each processing unit comprising a fluid inlet, a fluid outlet, and a fluid delivery device, where the first and second processing units are connected in series by at least one fluid connection with fluid flowing from outlet of one processing unit to inlet of a subsequent processing unit, and
   b. at least one means for introducing additional liquid to the fluid connection
   c. at least one means for removing excess liquid from the fluid connection
2. An apparatus according to embodiment 1, comprising
   a. at least two independent processing units, a first processing unit and a second processing unit, each processing unit comprising a fluid inlet, a fluid outlet, and a fluid delivery device, where the first and second processing units are connected in series by at least one fluid connection with fluid flowing from outlet of one processing unit to inlet of a subsequent processing unit, and
   b. at least one fluid inlet in the fluid connection between two processing units, providing a means for introducing additional liquid to the fluid connection
   c. at least one fluid outlet in the fluid connection between two processing units, providing a means for removing excess liquid from the fluid connection
3. An apparatus according to any of embodiments 1 or 2 wherein the inlet of said fluid connection has a flow direction restricted by a means of unidirectional flow control to restrict the liquid to flow unidirectionally towards the fluid connection between processing units.
4. An apparatus according to any of embodiments 1 to 3 wherein the outlet of said fluid connection has a flow direction restricted by a means of unidirectional flow control to restrict liquid to flow unidirectionally from the fluid connection between processing units.
5. An apparatus according to any of embodiments 3 to 4, wherein means of unidirectional flow is a check valve.
6. An apparatus according to any of the preceding embodiments, wherein at least one of the fluid delivery devices comprises a pump.
7. A method of recombinant protein production from a cell line comprising the steps of
   i. cultivating a cell line in a cultivation unit where an outflow of culture supernatant is provided by a pump connecting the cultivation unit to a purification unit using an apparatus defined in any of embodiments 1 to 5;
   ii. performing a protein purification on said purification unit with an feed inflow provided by a pump
8. A method according to embodiment 7 where the cell line is a mammalian cell line.
9. A method according to embodiments 7 or 8 where the protein is selected from the group consisting of coagulation factors, fusion proteins comprising a coagulation factor, insulin, GLP derivatives, GHs, receptors, and antibodies, including FAbs.
10. A method according to embodiment 9 where the protein is selected from the group consisting of a coagulation factor and an antibody.
11. A method according to any of claims 7 to 10, wherein the outflow of the cultivation unit matches the feed to the purification system
12. A method according to any of embodiments 7 to 11, wherein the protein purification is performed by means of chromatography, filtration or combinations thereof.
13. An apparatus comprising
   a. at least two independent processing units, a first and a second processing unit, each processing unit comprising a fluid inlet, a fluid outlet, and a fluid delivery device, where the first and the second processing units are connected in series by at least one fluid connection with fluid flowing from outlet of one processing unit to inlet of a subsequent processing unit;
   b. at least one fluid inlet in the fluid connection between two processing units
   c. at least one fluid outlet in the fluid connection between two processing units.
14. An apparatus according to embodiment 13 for separating a molecule of interest from a heterogeneous fluid mixture comprising
   a. at least two independent processing units, a first and a second processing unit, each processing unit comprising a fluid inlet, a fluid outlet, and a fluid delivery device, where the first and the second processing units are connected in series by at least one fluid connection with fluid flowing from outlet of one processing unit to inlet of a subsequent processing unit;
   b. at least one fluid inlet in the fluid connection between two processing units; and
   c. at least one fluid outlet in the fluid connection between two processing units.
15. An apparatus according to any of embodiments 13 or 14 wherein the inlet of said fluid connection has a flow direction restricted by a means of unidirectional flow control to restrict the liquid to flow unidirectionally towards the fluid connection between processing units
16. An apparatus according to any of embodiments 13 to 14 wherein the outlet said fluid connection has a flow direction restricted by a means of unidirectional flow control to restrict liquid to flow unidirectionally from the fluid connection between processing units.
17. An apparatus according to any of embodiments 15 to 16, wherein means of unidirectional flow is a check valve.
18. An apparatus according to any of embodiments 13 to 17, wherein the first processing unit is selected from the group consisting of a bioreactor, a fermentation unit, a tube reactor, an ultrafiltration unit, a homogenizer, a centrifuge unit, and a chromatography unit, each of which with a continuous or semi-continuous inflow, preferably a continuous inflow.
19. An apparatus according to any of embodiments 13 to 18, wherein the second processing unit is typically selected from the group consisting of an ultrafiltration unit, a homogenizer, a centrifuge unit, and a chromatography unit, each of which with a continuous or semi-continuous outflow, preferably a continuous outflow.
20. An apparatus according to any of embodiments 13 to 19, wherein the first processing unit is a cultivation unit with a continuous out-flow, such as a perfusion cultivation unit, and the second process unit is a chromatography system with a continuous in-flow.
21. An apparatus according to any of embodiments 13 to 19, wherein the first processing unit is an ultrafiltration unit with a continuous out-flow and the second process unit is a chromatography system with a continuous in-flow.
22. An apparatus according to any of embodiments 13 to 19, wherein the first processing unit is a chromatography system with a continuous or semi-continuous out-flow, such as an Äkta chromatography system and the second process unit is an ultrafiltration unit with a continuous in-flow.
23. An apparatus according to any of embodiments 13 to 19, wherein the first processing unit is a simulated moving bed chromatography system with a continuous out-flow, and the second process unit is a chromatography system with a continuous in-flow.
24. An apparatus according to any of embodiments 13 to 19, wherein the first processing unit is a chromatography system with a continuous or semi-continuous out-flow, such as an Äkta chromatography system, and the second process unit is a chromatography system with a continuous in-flow, such as an Äkta chromatography system.
25. An apparatus according to any of embodiments 13 to 19, wherein the first processing unit is a tube reactor with a continuous or semi-continuous out-flow and the second process unit is a chromatography system with a continuous in-flow, such as an Äkta chromatography system.
26. An apparatus according to any of embodiments 13 to 19, wherein the first processing unit is a homogenizer with a continuous out-flow and the second process unit is a continuous centrifuge with a continuous in-flow.
27. An apparatus according to any of embodiments 13 to 19, wherein the first processing unit is a continuous fermentation with a continuous out-flow and the second process unit is a continuous centrifuge with a continuous in-flow.
28. An apparatus according to any of embodiments 13 to 19, wherein the first processing unit is a continuous centrifuge with a continuous out-flow and the second process unit is a chromatography system with a continuous in-flow, such as an Äkta chromatography system.
29. A method for purifying a liquid containing at least one target molecule, comprising the use of an apparatus as defined in any one of embodiments 13 to 32.
30. A method for separating a target protein or proteins from a heterogeneous fluid mixture comprising
i. a first processing step comprising producing a fluid mixture containing the protein of interest
ii. transferring by means of a unidirectional flow the fluid mixture from an outlet of a first processing step to an inlet of a second processing step
iii. matching the flow rate of liquid delivered from first processing step to that of flow rate of liquid taken up in second processing step by removing surplus liquid or adding compatible liquid
iv. a second processing step comprising producing a further purified fluid mixture containing the protein of interest.
31. A method according to embodiments 30 wherein the first processing step is a continuous cultivation process producing clarified cell culture harvest fluid containing the protein of interest; and the second processing step is a continuous chromatography process producing partially purified fluid containing the target protein or proteins.
32. A process for the continuous or semi-continuous production of a purified protein, the process comprising the use of a an integrated apparatus comprising
a. a cell culture bioreactor with cell separation unit having continuous outflow
b. a means for at least partially purifying a protein with a continuous inflow, such as a means for performing chromatography or a means for filtration,
c. a device with two three-way connectors and two check valves for matching the outflow of the bioreactor and the inflow of the means for at least partially purifying a protein.

### EXAMPLES

### Example 1

### Integrated Continuous Production of B-Domain Deleted FVIII Variant

The process for producing a B-domain deleted FVIII variant (as described in Thim et al, Haemophilia (2010), 16, 349-359) was translated into an integrated continuous production set-up.

Briefly, a clonal Chinese hamster ovary (CHO) cell line expressing the FVIII variant was cultivated in a chemically defined animal component free medium. After propagation, the cell line was used to inoculate a 5 L stirred tank bioreactor with an ATF cell retention system operating in perfusion mode delivering an output of clarified cell harvest for purification. The bleed rate was manipulated to maintain a constant viable biomass using feedback control from an on-line capacitance probe.

The original batch-mode purification procedure including four chromatographic steps
a capture step on a Capto MMC column (GE HealthCare, Uppsala, Sweden)
an immunoaffinity chromatography step
an anionic exchange chromatography (Macro-Prep 25Q Support, BioRad Laboratories, Hercules, Calif., USA)
a gel filtration step (Superdex 200, GE HealthCare).
   was translated into a continuous purification procedure on an ÄKTA Pure chromatographic system (GE Healthcare). This was achieved by employing dual alternating capture columns followed by automated multi-step purification. Buffer exchange columns (Sephadex G-25, GE Healthcare) were introduced between steps 1-2 and 3-4 to replace manual dilutions hence increasing the number of columns to six. While one capture column is loaded with harvest, purification and cleaning takes place on the other capture column and the following chromatographic steps without any intermediate storage. In this way, the ÄKTA chromatographic system was converted into a system with a continuous input of clarified cell culture harvest and with a semi-continuous output delivering sub-batches of purified protein with a cycle time of ca 16 h.

An integrated continuous system for production of the B-domain deleted FVIII variant was obtained by connecting the outlet from the upstream unit (the ATF perfusion setup) to the inlet of the downstream unit (the ÄKTA system), see FIG. 5. The flow rate from the ATF is controlled by the harvest pump (h) and the flow rate to the ÄKTA is controlled by the sample application pump (j). For continuous operation, these flow rates need to be matched to avoid process failure due to overflow/overpressure or gas bubbles/under pressure. To resolve this without introducing any intermediate storage in the process flow, an arrangement with two three-way connections and two check valves was employed. If the flow delivered by the harvest pump (h) is lower than the flow taken by the sample application pump (j), the difference flow will be taken from the ATF system via the check valve (i). If the flow delivered from the harvest pump (h) is higher than that taken up by the sample application pump (j), the surplus flow will go through the check valve (l) to e.g. a collection vessel (m).

Results

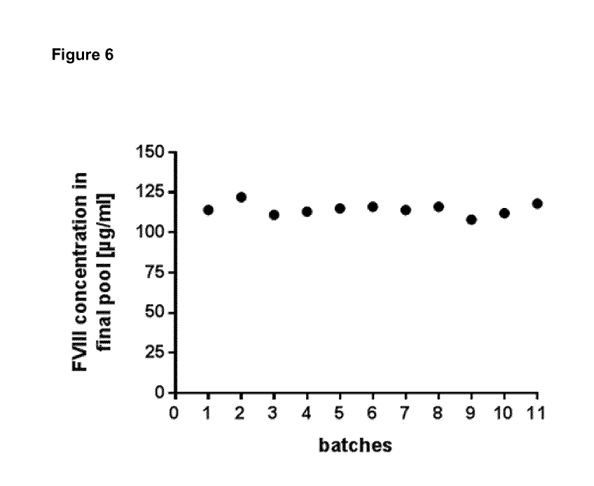

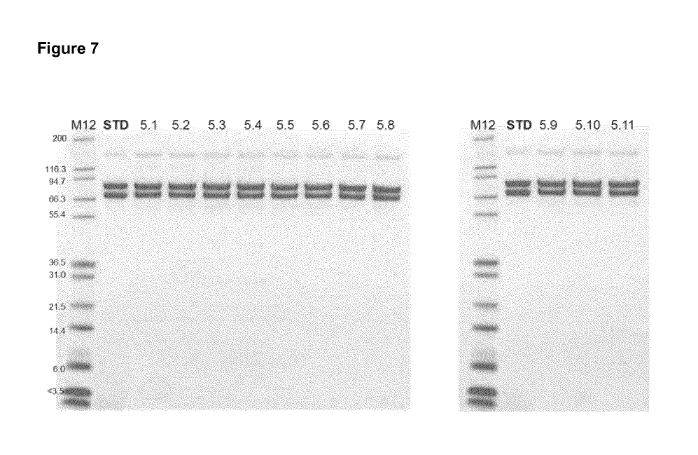

The ATF perfusion setup and the downstream ÄKTA system were connected using the apparatus of the invention. When connected both the ATF and the ÄKTA was individually and independently fine-tuned. This meant e.g. that the downstream ÄKTA was stopped and started without considerations to the upstream ATF perfusion setup without jeopardising the cell cultivation regarding aseptic run conditions and supply of fresh cell media. The integrated continuous production set-up was then evaluated between cultivation day 24 and cultivation day 31 in a perfusion cultivation system for expression of the B-domain deleted FVIII variant. The integrated system was deliberately stopped after one week of uninterrupted continuous operation corresponding to 11 purified sub-batches. The resulting pools from the final gel filtration step were measured by RP-HPLC and SDS-PAGE (FIGS. 6 and 7) and as can be seen the output is constant and consistent with respect to titre and quality. This demonstrates that the proposed set-up is capable of long-term integrated continuous operation at steady state.

In the batch-mode process with intermediate storage between steps, the normal processing time from clarified harvest to purified protein is at least around 4 days. In the presented integrated continuous process, the average processing time within a sub-batch is 16 h. The reduced processing time makes the integrated continuous approach well suited for fragile proteins prone to degradation. Compared to the batch-mode process, the integrated continuous process also reduces the lead time from start of cultivation to final purified protein with at least 3 days. Furthermore, the purification chromatograms provide information about the amount of purified protein in each sub-batch which provides a continuous monitoring of the integrated process. This can for instance be used to stop a campaign when enough protein has been produced (“just-enough” production) allowing the allowing the lead time to be reduced even further.

### Example 2

### Integrated Continuous Production of Dimeric Protein

An integrated continuous set-up for producing a dimer form of a recombinant protein was designed.

A clonal Chinese hamster ovary (CHO) cell line expressing the recombinant protein cultivated in a chemically defined animal component free medium. After propagation, the cell Line was used to inoculate a 5 L stirred tank bioreactor with an ATF cell retention system operating in perfusion mode delivering an output of clarified cell harvest for purification. The bleed rate was manipulated to maintain a constant viable biomass using feedback control from an on-line capacitance probe. The clarified harvest contains both monomer and dimer forms of the recombinant protein.

A continuous purification procedure including three chromatographic steps

an immunoaffinity chromatography step

an ion exchange chromatography step

a gel filtration step
was implemented on an ÄKTA Pure chromatographic system (GE Healthcare). This was achieved by employing dual alternating capture columns followed by automated multi-step purification. While one capture column is loaded with harvest, purification and cleaning takes place on the other capture column and the following chromatographic steps without any intermediate storage. In this way, the ÄKTA chromatographic system was converted into a system with a continuous input of clarified cell culture harvest and with a semi-continuous output delivering sub-batches of purified protein with a cycle time of ca 18 h.

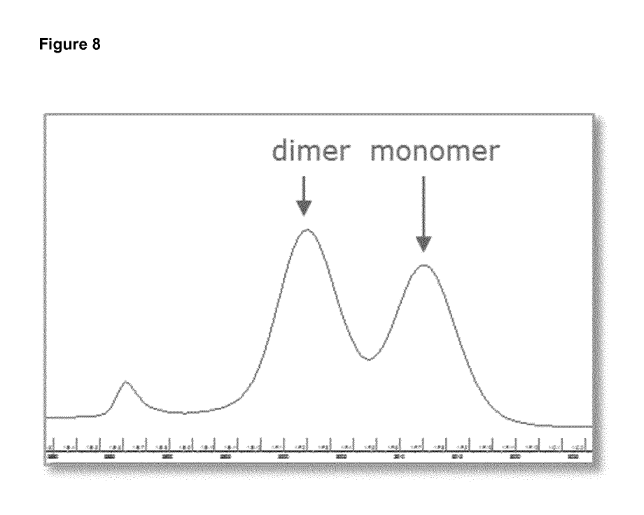

An integrated continuous system for production of the dimer form of the recombinant protein was obtained by connecting the outlet from the upstream unit (the ATF perfusion setup) to the inlet of the downstream unit (the ÄKTA system), as described in Example 1 however without the need for chromatography columns 4-6. Importantly, the UV absorbance chromatograms from the final gel filtration step, see example in FIG. 8, can be used to monitor the amount of purified dimer as well as the ratio between dimer and monomer or the dimer fraction, e.g. by integration of the areas under the dimer and monomer peaks.

Results

The ATF perfusion setup and the downstream ÄKTA system were connected using the three-way connector unit. When connected both the ATF and the ÄKTA was individually and independently fine-tuned. This meant e.g. that the downstream ÄKTA was stopped and started without considerations to the upstream ATF perfusion setup without jeopardising the cell cultivation regarding aseptic run conditions and supply of fresh cell media. The integrated continuous production set-up was then evaluated between cultivation day 18 and cultivation day 29 in a perfusion cultivation for expression of the recombinant protein. During this period, there was a change in desired operating point in the cultivation (increase of viable biomass). The integrated system was deliberately stopped after 15 purified sub-batches corresponding to ca 11 days.

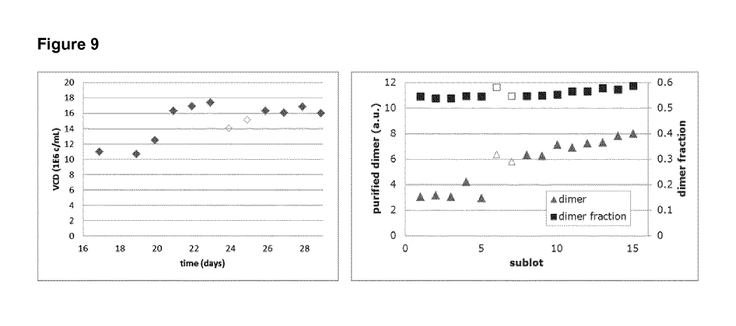

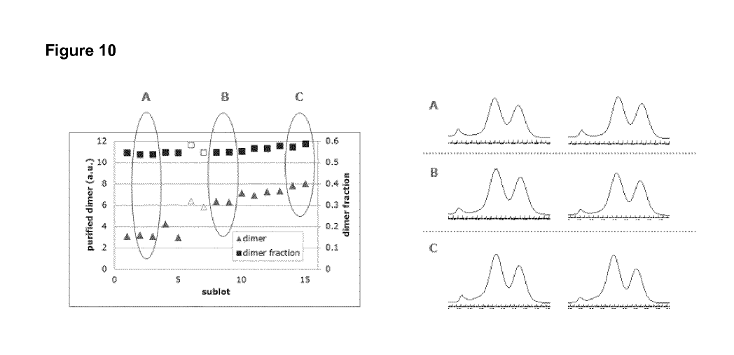

When the viable biomass increases, there is a concomitant increase in the amount of purified dimer as estimated from the gel filtration chromatograms (see FIG. 9). Furthermore, there is an indication of an increase of the dimer fraction due to the change in the cultivation. Visual inspection of individual chromatograms, before, during and after the change suggest that there is indeed an increased dimer fraction due to the change in the cultivation (see FIG. 10)

Again, the example demonstrates that the proposed set-up is capable of long-term integrated continuous operation. In particular, the example further demonstrates the monitoring capability of the integrated system by following process changes and detecting changes in product quality attributes.

### Example 3

### Integrated Continuous Capture of FVII Variant

An integrated continuous set-up for cultivation and capture of a recombinant FVII variant was designed.

A Chinese hamster ovary (CHO) cell line expressing a FVII variant was cultivated in a chemically defined animal component free medium. After propagation, the cell line was used to inoculate a 15 L stirred tank bioreactor with an ATF cell retention system operating in perfusion mode delivering an output of clarified cell harvest for purification. Steady-state in viable biomass was achieved by a constant cell bleed rate.

A continuous capture procedure based on immunoaffinity chromatography was implemented on an ÄKTA Pure chromatographic system (GE Healthcare) employing dual alternating capture columns. While one capture column is loaded with harvest, purification and cleaning takes place on the other capture column. In this way, the ÄKTA chromatographic system was converted into a system with a continuous input of clarified cell culture harvest and with a semi-continuous output delivering sub-batches of captured protein with a cycle time of ca 24 hours.

An integrated continuous system for cultivation and capture of a FVII variant was obtained by connecting the outlet from the upstream unit (the ATF perfusion setup) to the inlet of the downstream unit (the ÄKTA system), as described in Example 1 however without the need for chromatography columns 2-6.

FVII variant titer in the cultivation and the ATF harvest was measured by affinity HPLC. FVII variant titer in capture pools was measured by SE-HPLC.

Results

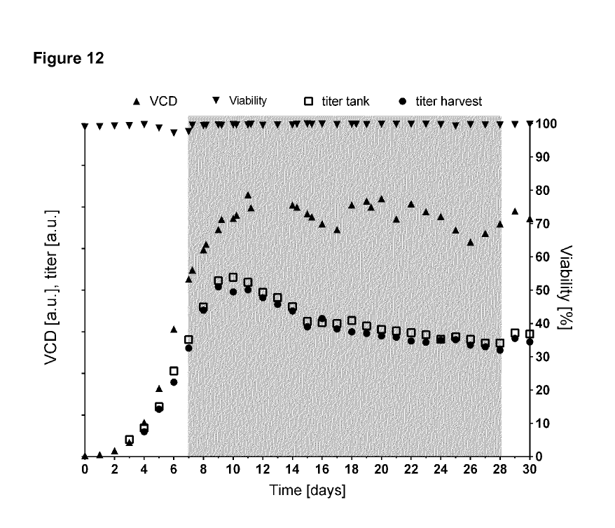

The integrated continuous production set-up was evaluated between cultivation day 7 and cultivation day 28 in a perfusion cultivation for expression of a FVII variant (see FIG. 12). The integrated system was deliberately stopped after 21 capture sub-batches corresponding to 21 days.

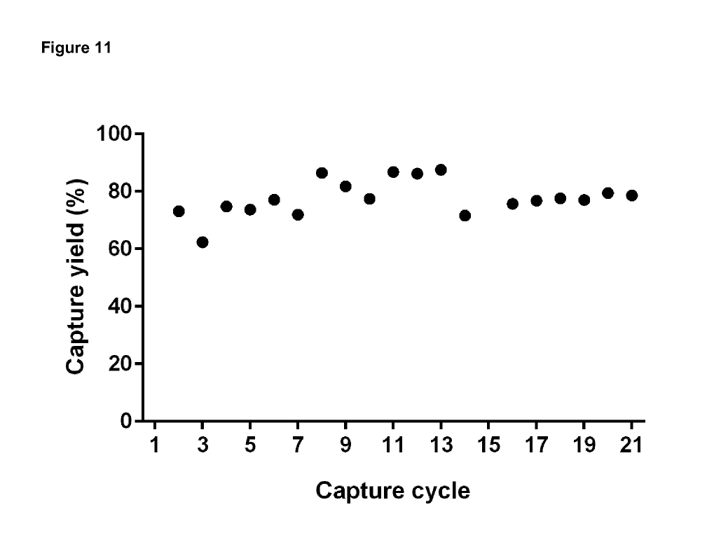

The capture yield varies slightly around an average of 74% but there are no trends or other indications of decreasing performance with time (FIG. 11). The example further demonstrates that the proposed set-up is capable of long-term integrated continuous operation.

### Example 4

### Integrated Continuous Production of Monoclonal Antibody

An integrated continuous set-up for producing a monoclonal antibody was designed.

A clonal CHO cell line expressing a monoclonal antibody of IgG4 format was cultivated in a chemically defined animal component free medium. After propagation, the cell line was used to inoculate a 5 L stirred tank bioreactor with an ATF cell retention system operating in perfusion mode delivering an output of clarified harvest for purification. The temperature set-point was changed after 7 days of cultivation from 36.5° C. to 32° C. to reduce cell growth and cell bleed with a constant rate was started.

A continuous purification procedure including a single Protein A affinity chromatography step was implemented on an ÄKTA Explorer chromatographic system (GE Healthcare). This was achieved by employing dual alternating capture columns. In this way, the ÄKTA chromatographic system was converted into a system with a continuous input of clarified cell culture harvest and with a semi-continuous output delivering sub-batches of purified protein with a cycle time of ca 2.5 h.

An integrated continuous system for production of the monoclonal antibody was obtained by connecting the upstream unit (the ATF perfusion setup) to the downstream unit (the ÄKTA system) using the apparatus illustrated in FIG. 4. The integration is described in detail in Example 1.

Results

The integrated continuous production set-up was evaluated for a short production run on cultivation day 8 of the perfusion cultivation. Three sub-batches were produced to test the system and evaluate the robustness of the system to changes in flow rates on the purification system. To facilitate this, the three consecutive sub-batches were run using inflow rates on the ÄKTA system corresponding to 100% (sub-batch A), 75% (sub-batch B), and 125% (sub-batch C), respectively, of the initial flow rate of 2.5 mL/min on the ÄKTA system.

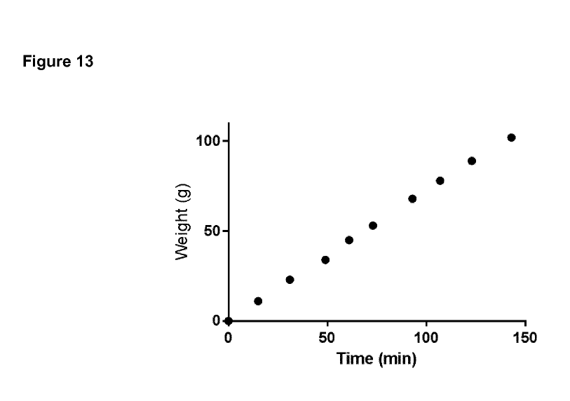

It was observed, that the outflow of the perfusion system almost matched the inflow of the purification system at the 100% setting. The flow rates were 2.59 mL/min and 2.5 mL/min for perfusion outflow and purification inflow, respectively, and a small amount of liquid could be observed in the collection tank. When the inflow of the ÄKTA system was reduced to 75%, perfusion harvest was flowing to the collection tank at a rate of 0.72 g/min (0.63 g/min expected at perfect match at 100% ÄKTA flow), see FIG. 13. This illustrates the capability of the integration device to maintain constant flows on both connected systems where outflow of the first system is higher than inflow of the second system.

To investigate if the integration device is suitable in the inverted situation, the ÄKTA system inflow was increased to 125% of the starting value. As a consequence the total dilution rate of the perfusion system increased.

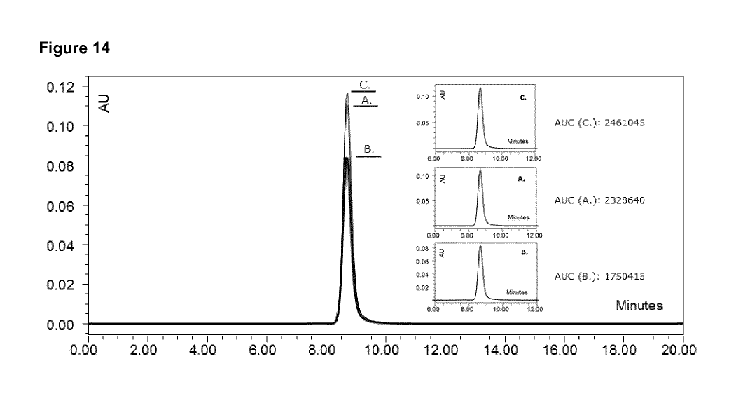

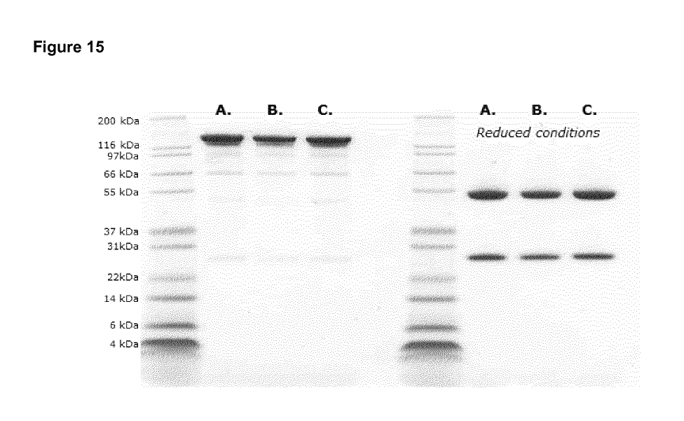

SE-HPLC chromatograms for quantification of the purified sub-batches are shown in FIG. 14. A decrease in the AUC from sub-batch A to sub-batch B can be observed, which correlates with a decrease in volume load due to the decrease in pump rate on the ÄKTA system. Comparing the AUC from sub-batch A and sub-batch C, an increase can be observed. This increase is lower than the increase in volume load, which can be attributed to the reduction of harvest titer due to increased dilution rate in the perfusion system. Product quality for all sub-batches was comparable as illustrated in FIG. 15.

This shows that the integration device is able to match the flow of two independently operated continuous unit operations. Furthermore it demonstrates the robustness to flow variations for a continuous production setup using the integration device.

### Example 5

### Integrated Continuous Capture of Insulin Precursor

An integrated continuous set-up for producing an insulin precursor was designed.

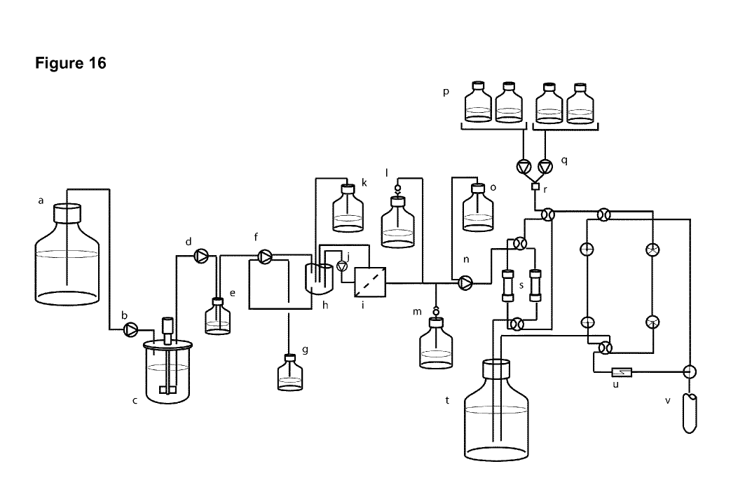

A recombinant Saccharomyces cerevisiae strain expressing an insulin precursor was grown in a normal yeast growth medium (glucose, yeast extract, salts, and vitamins) in a continuous cultivation setup in 0.3 L laboratory bioreactor at aerobic conditions. To maintain a constant volume in the bioreactor culture broth is pumped out frequently and led to a buffer flask from which broth is continuously pumped into a microfiltration set up for cell separation. After removing the cells the flow is transported through the apparatus of the invention before pH is brought down by employing in-line dilution and the cell-free harvest is applied to a CIEX capture column. To be able to handle a continuous flow of cell-free harvest a dual alternating capture columns including SP Sepharose FF (GE Healthcare) were used. When one of the capture columns is loaded the other one is washed, eluted and re-equilibrated, and then they switch, and switch again, repeatedly. The whole setup can be seen in FIG. 16.

The aim of the experiments were (1.) to verify that it is doable to produce an insulin precursor continuously and integrated and (2.) to assess if apparatus of the invention can counteract unwanted variations in the flow between the cell separation device (cross-flow filtration device) and the purification setup (modified ÄKTA explorer)

Results:

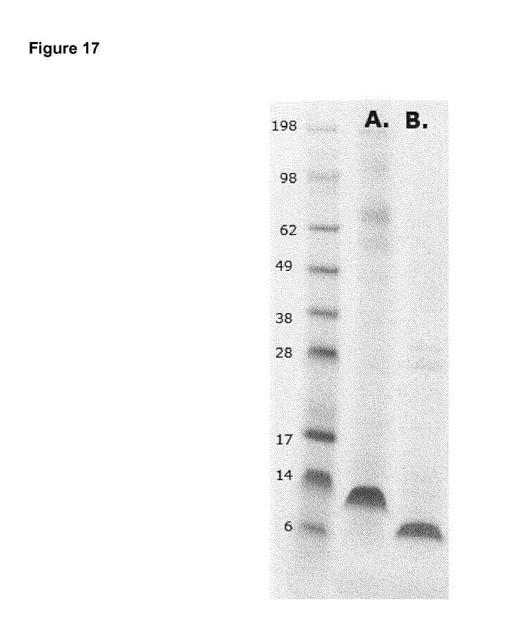

To verify that it is possible to produce an insulin precursor continuously and with integrated up-stream and down-stream devises the recovered peptides were treated with the lysine-specific ALP enzyme and analysed with SDS-PAGE (FIG. 17). The SDS-PAGE shows what was expected; the insulin precursor is matured by ALP.

When conduction the test of the setup depicted in FIG. 1 the buffer flask with check valve 1 (see the legend of FIG. 16) and the surplus collection flask with check valve 2 (legend item m in FIG. 16) were put on balances and their weight was monitored.

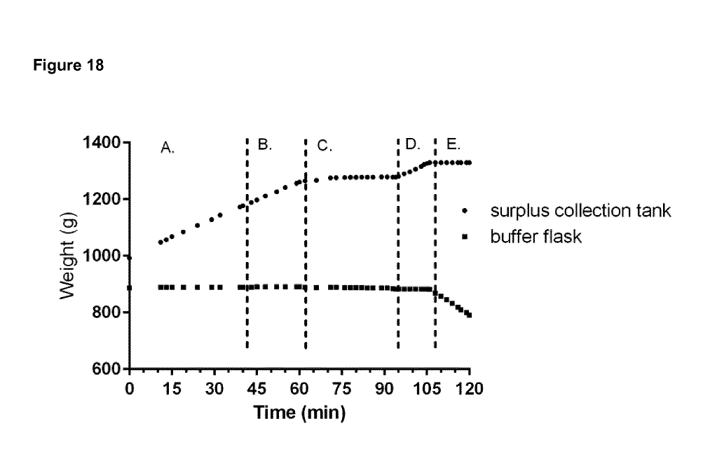

In FIG. 18, in time period A, the ÄKTA pump was set to a value believed to correspond to the out-flow flow from the cell separations device. It was though shown that the flow from the cell separation device was much higher as the surplus was led into the collection vessel detected by the increasing weight. In time period B the ÄKTA flow was increased to 150% but still surplus liquid was collected in the surplus collection flask. In time period C, at 200% of the initial ÄKTA flow, the flow of surplus liquid into the surplus collection vessel stopped indicating equal flow out from the cell-separation device and in to the purification setup. At time interval D the ÄKTA was paused resulting that the liquid was directly led into the collection vessel and at interval E the cross-flow filtration device was stopped resulting that liquid was only drawn from the buffer flask. During this 2 hour trial both the cross-flow device and purification apparatus kept on working un-disturbed even though forced miss-matches in flow rates were applied.

The cross-filtration contraption to some extent alters the flow through its membrane when the pressure on the permeate side changes, hence when the ÄKTA flow is changed. That means that it was only expected to have liquid drawn out from the buffer flask (legend item I; FIG. 16) when there was no flow out from the cross-flow filtration membrane, which was observed.

1. An apparatus comprising
a. at least two independent processing units, a first processing unit and a second processing unit, each processing unit comprising a fluid inlet, a fluid outlet, and a fluid delivery device, where the first and second processing units are connected in series by at least one fluid connection with fluid flowing from outlet of one processing unit to inlet of a subsequent processing unit, and
b. at least one means for introducing additional liquid to the fluid connection
c. at least one means for removing excess liquid from the fluid connection

2. An apparatus according to claim 1, comprising
a. at least two independent processing units, a first processing unit and a second processing unit, each processing unit comprising a fluid inlet, a fluid outlet, and a fluid delivery device, where the first and second processing units are connected in series by at least one fluid connection with fluid flowing from outlet of one processing unit to inlet of a subsequent processing unit, and
b. at least one fluid inlet in the fluid connection between two processing units, providing a means for introducing additional liquid to the fluid connection
c. at least one fluid outlet in the fluid connection between two processing units, providing a means for removing excess liquid from the fluid connection

3. An apparatus according to claim 1, wherein the inlet of said fluid connection has a flow direction restricted by a means of unidirectional flow control to restrict the liquid to flow unidirectionally towards the fluid connection between processing units.

4. An apparatus according to claim 1, wherein the outlet of said fluid connection has a flow direction restricted by a means of unidirectional flow control to restrict liquid to flow unidirectionally from the fluid connection between processing units.

5. An apparatus according to claim 1, wherein at least one of the fluid delivery devices comprises a pump.

6. A method of recombinant protein production from a cell line comprising the steps of
a. cultivating a cell line in a cultivation unit where an outflow of culture supernatant is provided by a pump connecting the cultivation unit to a purification unit using an apparatus defined in claim 1;
b. performing a protein purification on said purification unit with a feed inflow provided by a pump.

7. A method according to claim 6, wherein the protein is selected from the group consisting of coagulation factors, fusion proteins comprising a coagulation factor, insulin, GLP derivatives, GHs, receptors, and antibodies, including FAbs.

8. A method according to claim 6, wherein the outflow of the cultivation unit matches the feed to the purification system.

9. An apparatus for separating a molecule of interest from a heterogeneous fluid mixture comprising
a. at least two independent processing units, a first and a second processing unit, each processing unit comprising a fluid inlet, a fluid outlet, and a fluid delivery device, where the first and the second processing units are connected in series by at least one fluid connection with fluid flowing from outlet of one processing unit to inlet of a subsequent processing unit;
b. at least one fluid inlet in the fluid connection between two processing units; and
c. at least one fluid outlet in the fluid connection between two processing units.

10. An apparatus according to claim 9 wherein the inlet of said fluid connection has a flow direction restricted by a means of unidirectional flow control to restrict the liquid to flow unidirectionally towards the fluid connection between processing units

11. An apparatus according to claim 9, wherein the first processing unit is selected from the group consisting of a bioreactor, a fermentation unit, a tube reactor, an ultrafiltration unit, a homogenizer, a centrifuge unit, and a chromatography unit, each of which with a continuous or semi-continuous inflow.

12. An apparatus according to claim 9, wherein the second processing unit is typically selected from the group consisting of an ultrafiltration unit, a homogenizer, a centrifuge unit, and a chromatography unit, each of which with a continuous or semi-continuous outflow.

13. An apparatus according to claim 9, wherein the first processing unit is a cultivation unit with a continuous out-flow, and the second process unit is a chromatography system with a continuous in-flow.

14. A method for separating a target protein or proteins from a heterogeneous fluid mixture, comprising
a first processing step comprising producing a fluid mixture containing the protein of interest,
transferring by means of a unidirectional flow the fluid mixture from an outlet of a first processing step to an inlet of a second processing step,
matching the flow rate of liquid delivered from first processing step to that of flow rate of liquid taken up in second processing step by removing surplus liquid or adding compatible liquid, and a second processing step comprising producing a further purified fluid mixture containing the protein of interest.

15. A method according to claim 14 wherein the first processing step is a continuous cultivation process producing clarified cell culture harvest fluid containing the protein of interest; and the second processing step is a continuous chromatography process producing partially purified fluid containing the target protein or proteins.

16. An apparatus according to claim 9, wherein the first processing unit is selected from the group consisting of a bioreactor, a fermentation unit, a tube reactor, an ultrafiltration unit, a homogenizer, a centrifuge unit, and a chromatography unit, each of which with a continuous inflow.

17. An apparatus according to claim 9, wherein the second processing unit is typically selected from the group consisting of an ultrafiltration unit, a homogenizer, a centrifuge unit, and a chromatography unit, each of which with a continuous outflow.

18. An apparatus according to claim 13, wherein the first processing unit is a perfusion cultivation unit.
